(19) United States
(12) Patent Application Publication
(10) Pub. No.: US 2014/0044809 A1
(43) Pub. Date: Feb. 13, 2014

(54) ANTACID FORMULATIONS AND ASSOCIATED METHODS

(71) Applicant: Elliott F. Brainard, Mammoth Lakes, CA (US)

(72) Inventor: Elliott F. Brainard, Mammoth Lakes, CA (US)

(21) Appl. No.: 13/758,772

(22) Filed: Feb. 4, 2013

Related U.S. Application Data
(63) Continuation-in-part of application No. PCT/US2011/046602, filed on Aug. 4, 2011.
(60) Provisional application No. 61/370,762, filed on Aug. 4, 2010.

(51) Int. Cl. A61K 36/45; A61K 31/194; A61K 36/73; A61K 36/185

(52) U.S. Cl. CPC A61K 36/45; A61K 36/185; A61K 31/194; A61K 36/73
USPC 424/732; 514/574

(57) ABSTRACT

The present invention provides antacid compositions and associated methods. In one aspect for example, an antacid composition suitable for delivery to a subject for reducing a digestive condition includes an effective amount of at least one citrate compound, an effective amount of blueberry product, and an effective amount of fruit extract including a member selected from the group consisting of pomegranate extract, bilberry extract, elderberry extract, blueberry extract, and combinations thereof.

### PRIORITY DATA

This application is a continuation-in-part of PCT Application No. PCT/US2011/046602, filed on Aug. 4, 2011, which claims benefit of U.S. Provisional Patent Application Ser. No. 61/370,762 filed on Aug. 4, 2010, each of which is incorporated herein by reference.

### BACKGROUND OF THE INVENTION

Heartburn, reflux, indigestion, and sour stomach are terms that refer to digestive upset conditions that are very common problems experienced by people throughout the world. Such conditions can be characterized by pain in the upper abdomen and upper abdominal fullness when eating, as well as sometimes being accompanied by bloating, belching, and/or nausea. In some cases, indigestion can be a result of gastroesophageal reflux disease or gastritis. Also, certain medications, such as calcium antagonists, nitrates, corticosteroids and non-steroidal anti-inflammatory drugs, and the like, can cause indigestion.

Antacids can be utilized for the temporary relief of digestive disorders. An antacid is a substance, generally a base or basic salt, which neutralizes stomach acidity. Thus, antacids perform a neutralization reaction, i.e. they buffer gastric acid, raising stomach pH to reduce acidity. Many antacids, however, contain ingredients that may cause adverse effects, particularly with prolonged usage. For example, regular high doses of calcium carbonate may cause alkalosis, which in turn may result in altered excretion of other drugs, and kidney stones. Also, a chemical reaction between calcium carbonate and hydrochloric acid may in some cases produce carbon dioxide gas, resulting in gastric distension that may not be well tolerated. Carbon dioxide formation can also lead to headaches and decreased muscle flexibility. As another example, recurrent high dosage use of antacids containing aluminum hydroxide may lead to the formation of insoluble aluminum-phosphate-complexes, with a risk for hypophosphatemia and osteomalacia. Although aluminum has a low gastrointestinal absorption, accumulation may occur in the presence of renal insufficiency. Also, aluminum-containing drugs may cause constipation.

Side effects from antacids vary depending on individual and other medications the individual may be taking. The most common side effects include changes in bowel functions, such as diarrhea, constipation, or flatulence.

### SUMMARY OF THE INVENTION

The present invention provides antacid compositions and associated methods. In one aspect for example, an antacid composition suitable for delivery to a subject for reducing a digestive condition includes an effective amount of at least one citrate compound, an effective amount of a blueberry product such as blueberry fiber, blueberry fruit, or a combination thereof, and an effective amount of fruit extract including at least one of a pomegranate extract, a bilberry extract, an elderberry extract, a blueberry extract, and combinations thereof. Non-limiting examples of citrate compounds can include magnesium citrate, calcium citrate, sodium citrate, potassium citrate, and the like, including combinations thereof. In one aspect, the citrate compound can include magnesium citrate and calcium citrate.

The citrate compound can be included in the composition in any amount that is an effective amount. For example, in some aspects the composition can be formulated as a solid dosage form, such as a tablet, a capsule, a powder, a gum, an effervescent tablet, a soft chew, and combinations thereof, and where the blueberry product is blueberry fiber. In one such aspect, the effective amount of citrate compound is from about 1.0% w/w to about 75.0% w/w. In yet another aspect, the citrate compound includes magnesium citrate and calcium citrate, the effective amount of magnesium citrate is from about 0.5% w/w to about 15.0% w/w, and the effective amount of calcium citrate is from about 0.5% w/w to about 40.0% w/w.

In another aspect, a solid dosage form can include a fruit extract having at least two members selected from the group consisting of pomegranate extract, bilberry extract, elderberry extract, blueberry extract, and combinations thereof. In yet another aspect, the fruit extract includes pomegranate extract, bilberry extract, and elderberry extract. The fruit extract can be included in the composition in an amount of from 0.5% w/w to about 15.0% w/w. Additionally, the in one aspect the effective amount of blueberry fiber is from about 1.0% w/w to about 75.0% w/w.

In another aspect, the composition can be formulated as a liquid dosage form such as, for example, a liquid, a syrup, an elixir, a suspension, an effervescent beverage, and combinations thereof, and were the blueberry product is blueberry fruit. In one such aspect, the effective amount of citrate compound is from about 0.1% w/v to about 20% w/v. In another aspect, the citrate compound includes magnesium citrate and calcium citrate, the effective amount of magnesium citrate is from about 0.05% w/v to about 10% w/v, and the effective amount of calcium citrate is from about 0.1% w/v to about 20% w/v. In yet another aspect, the effective amount of blueberry fruit is from about 0.1% w/v to about 10% w/v. In a further aspect, the effective amount of fruit extract is from about 0.04% w/v to about 10% w/v. In some aspects, either of the solid or liquid dosage forms can include vitamin D or vitamin D3.

The present invention additionally provides antacid compositions suitable for delivery to a subject formulated as a chewable tablet. Such a composition can include a therapeutically effective amount of magnesium citrate, a therapeutically effective amount of calcium citrate, an effective amount of blueberry fiber, an effective amount of pomegranate extract, an effective amount of bilberry extract, an effective amount of elderberry extract, and an effective amount of blueberry extract.

In another aspect, an antacid composition suitable for delivery to a subject for reducing a digestive condition can include an effective amount of at least one citrate compound, an effective amount of a Saskatoon berry product such as Saskatoon berry fiber, Saskatoon berry fruit, or a combination thereof, and an effective amount of fruit extract including at least one of a pomegranate extract, a bilberry extract, an elderberry extract, a Saskatoon berry extract, and combinations thereof.

The present invention additionally provides methods for using antacid compositions. In one aspect, for example, a method of treating a digestive condition is provided, comprising orally ingesting a composition according to aspects of the present invention.

In a further aspect, the present invention provides an antacid composition suitable for delivery to a subject for reducing a digestive condition, comprising an effective amount of at least two citrate compounds.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION OF THE INVENTION

Definitions

In describing and claiming the present invention, the following terminology will be used in accordance with the definitions set forth below.

The singular forms “a,” “an,” and “the” include plural referents unless the context clearly dictates otherwise. Thus, for example, reference to “a juice” includes reference to one or more of such juices, and reference to “the tablet” includes reference to one or more of such tablets.

As used herein, “subject” refers to a mammal that may benefit from the administration of a composition or method of this invention. Examples of subjects include humans, and may also include other animals such as horses, pigs, cattle, dogs, cats, rabbits, and the like, including aquatic mammals.

As used herein, “digestive condition” and “digestive disorder” can be used interchangeably, and refer to numerous conditions of the digestive system. Such conditions can include, without limitation, feelings between the stomach and the esophagus of pain, discomfort, bloating, and the like. In one aspect, a digestive condition is a condition for which a traditional antacid may provide some relief.

As used herein, “formulation” and “composition” may be used interchangeably and refer to a combination of elements that is presented together for a given purpose. Such terms are well known to those of ordinary skill in the art.

As used herein, “effective amount” refers to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “administration,” and “administering” refer to the manner in which a composition is presented to a subject. Administration can be accomplished by various routes well-known in the art including oral and non-oral methods.

“Oral administration” can be achieved by swallowing, chewing, or sucking of an oral dosage form of a composition. Examples of well known oral dosage forms include tablets, capsules, caplets, powders, granulates, liquids, beverages, syrups, elixirs, confections, or other food items, etc.

As used herein, “extract” when used in connection with a plant or plant part, refers to material that has been removed from the plant source, or a portion thereof, including the flower, fruit, seed, peel, leaf, root, bark, stem, etc. As will be recognized by those of ordinary skill in the art, extracts may be either crude or refined to a selected degree in order to isolate specified materials. Extracts can take a variety of forms including powders, juices, purees, etc. A number of extraction processes that can be employed to produce the compositions of various types will be recognized by those skilled in the art, such as dehydration, lyophilization, etc.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained.

The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### THE INVENTION

The present invention encompasses compositions and associated methods for relieving digestion-related conditions such as heartburn, indigestion, and the like. The inventor has developed natural formulations that may provide relief from some digestion-related conditions, as well as providing various nutritional benefits. In one aspect for example, an antacid composition suitable for delivery to a subject for reducing a digestive condition can include an effective amount of at least one citrate compound and various fruit derived ingredients. For example, one useful ingredient can include an effective amount of a blueberry product, for example, blueberry fiber, blueberry fruit, blueberry extract, and the like. The composition can also include an effective amount of fruit extract, which can include a variety of fruit extracts, including for example, at least one of a pomegranate extract, a bilberry extract, an elderberry extract, a blueberry extract, a Saskatoon berry extract, and the like. In some aspects, reducing a digestive condition can include reducing acid in the upper G.I. tract.

A variety of citrate compounds are contemplated for inclusion in the present compositions. It should be noted that any form of citrate, including various salts thereof, that can be safely delivered to the subject and provide compositional benefits according to aspects of the present invention are considered to be within the present scope. Non-limiting examples can include magnesium citrate, calcium citrate, sodium citrate, potassium citrate, and combinations thereof. It should be noted that a salt of a given citrate compound can include all known salts of that compound. For example, the scope of the term sodium citrate should include monosodium citrate, disodium citrate, trisodium citrate, and the like. In a more specific aspect, the citrate compound can include at least one of magnesium citrate or calcium citrate. In another more specific aspect, the citrate compound includes magnesium citrate and calcium citrate. In one aspect, the present invention provides an antacid composition suitable for delivery to a subject for reducing a digestive condition, including an effective amount of at least two citrate compounds. Such a composition can be formulated with or without fruit extracts or fruit fiber.

The amount of citrate that can be included in the composition can vary depending on the dosage form of the composition, the intended dosage, presence of other ingredients in the composition, and the intended results of delivery of the composition. In one aspect for example, the composition can include an effective amount of citrate compound. An effective amount of citrate compound might be for example, the amount of citrate compound in the composition that would achieve a desired result. Such result could relate to the stability of the formulation, taste of the formulation, effect of the formulation such as relieving a digestive condition, and the like. It should be noted that in cases where multiple citrate compounds are utilized, an effective amount of citrate compound can be, in one aspect, an effective amount of each citrate compound, or in another aspect, an effective amount of the combination of citrate compounds.

In some aspects, the amount of citrate compound can be described for a solid dosage form, such as, for example, a tablet. In one aspect, the amount of citrate compound in the composition in such cases can be from about 1.0% w/w to about 75.0% w/w. In another aspect, the amount of citrate compound can be from about 1.0% w/w to about 50.0% w/w. In a further aspect, the amount of citrate compound can be from about 1.0% w/w to about 35.0% w/w. In yet a further aspect, the amount of citrate compound can be from about 1.0% w/w to about 20.0% w/w. In yet another aspect, the effective amount of citrate compound can be from about 0.5% w/w to about 5.0% w/w. It should be noted that this amount of citrate compound can describe a single citrate compound or multiple citrate compounds in the composition. In some aspects, more than one citrate compound can be included in the composition. As an example, a solid dosage form of an antacid composition can include magnesium citrate and calcium citrate. For such a case, in one aspect the amount of magnesium citrate is from about 0.5% w/w to about 15.0% w/w, and the amount of calcium citrate is from about 0.5% w/w to about 40.0% w/w. In another aspect, the amount of magnesium citrate is from about 0.5% w/w to about 5.0% w/w, and the amount of calcium citrate is from about 1.0% w/w to about 10.0% w/w. In yet another aspect, the amount of magnesium citrate is from about 0.5% w/w to about 2.0% w/w, and the amount of calcium citrate is from about 1.0% w/w to about 5.0% w/w. In one specific aspect, the amount of magnesium citrate is about 1.0% w/w, and the amount of calcium citrate is about 3.0% w/w. In some aspects, the amount of calcium citrate in the composition can be less than or equal to about 75% w/w. In other aspects, the amount of magnesium citrate in the composition can be less than or equal to about 50% w/w.

In other aspects, the amount of citrate compound can be described for a liquid dosage form, such as, for example, a drinkable liquid. In such cases, the amount of citrate compound in the composition can be from about 0.1% w/w to about 20.0% w/v. In another aspect, the amount of citrate compound can be from about 0.3% w/w to about 10.0% w/w. In a further aspect, the amount of citrate compound can be from about 0.3% w/w to about 5.0% w/w. In yet a further aspect, the amount of citrate compound can be from about 0.5% w/w to about 2.0% w/w. It should be noted that this amount of citrate compound can describe a single citrate compound or multiple citrate compounds in the composition. In some aspects, more than one citrate compound can be included in the composition. As an example, a liquid dosage form of an antacid composition can include magnesium citrate and calcium citrate. For such a case, in one aspect the amount of magnesium citrate is from about 0.05% w/w to about 10.0% w/w, and the amount of calcium citrate is from about 0.1% w/w to about 20.0% w/w. In another aspect, the amount of magnesium citrate is from about 0.1% w/w to about 5.0% w/w, and the amount of calcium citrate is from about 0.3% w/w to about 10.0% w/w. In yet another aspect, the amount of magnesium citrate is from about 0.1% w/w to about 2.0% w/w, and the amount of calcium citrate is from about 0.3% w/w to about 5.0% w/w. In one specific aspect, the amount of magnesium citrate is about 0.3% w/w, and the amount of calcium citrate is about 0.8% w/w.

As has been described, one useful ingredient of the present advanced compositions includes a blueberry product, such as blueberry fiber, blueberry fruit, blueberry extract, and the like. The blueberry product can impart various beneficial properties to the composition, including properties associated with the manufacture of the composition, bulk of the composition, color or taste of the resulting composition, as well as assisting in relief of the digestive condition. In one aspect, the antacid formulation is a solid dosage form and the blueberry product is blueberry fiber. It is intended that blueberry fiber refer to a solid or semisolid extract from blueberry fruit, although in some cases liquid such as juice may remain in the extract due to the manufacturing process. As such, in one aspect the blueberry fiber can include both fruit solids and liquid juice. In another aspect, blueberry fiber can include substantially solid fruit material remaining following liquid juice extraction from blueberries. Thus depending on the manufacturing protocol, the blueberry fiber can either be substantially devoid of juice or contain varying amounts of juice.

In one non-limiting aspect, for example, blueberry fiber can be derived by drying blueberry pomace. Blueberry pomace is the solid remains of blueberry fruit after juice removal by pressing. Pomace can be dried following juice extraction or can be further processed by mechanically sifting to eliminate impurities prior to drying. Thus in some cases blueberry fiber can be a finely milled solid product.

That being said, varying amounts of blueberry fiber are contemplated for inclusion in the compositions of the present invention. In one aspect for example, the composition can include an effective amount of blueberry fiber. The effective amount of blueberry fiber can include any amount to obtain a given result. Such a given result can be associated with formulating a manufacturing the composition, taste and/or texture of the resulting composition, reduction in a digestive condition, and the like.

In one aspect, the amount of blueberry fiber in the composition in such cases is from about 1.0% w/w to about 75.0% w/w. In another aspect, the amount of blueberry fiber is from about 5.0% w/w to about 40.0% w/w. In yet another aspect, the amount of blueberry fiber is from about 10.0% w/w to about 25.0% w/w. In one specific aspect, the amount of blueberry fiber is about 20.0% w/w. In another specific aspect, the amount of blueberry fiber is about 35.0% w/w. One specific non-limiting example of blueberry fiber is Milled Blueberry Fiber manufactured by Artemis International, Fort Wayne, Ind.

In other aspects, the antacid formulation is a liquid dosage form and the blueberry product is blueberry fruit. Blueberry fruit can be the fruit of a blueberry that is powdered, liquefied, frozen, fresh, concentrated, and the like. Accordingly, the amount of blueberry fruit can be described for a liquid dosage form, such as, for example, a drinkable liquid. One specific example of blueberry fruit can be blueberry powder, available from HerbaSway®, Wallingford, Conn. In one aspect, the amount of blueberry fruit in the composition can be from about 0.1% w/v to about 10% w/v. In another aspect, the amount of blueberry fruit in the composition can be from about 0.1% w/v to about 2% w/v. In one specific aspect, the amount of blueberry fruit in the composition can be about 0.2% w/v.

Fruit extracts can be included in the present compositions for a variety of reasons, such as flavorants, colorants, adding bulk, nutrition, alleviating digestive conditions, etc. As has been described, the present composition can include a fruit extract of at least one of pomegranate extract, bilberry extract, elderberry extract, blueberry extract, Saskatoon berry extract, and the like. In another aspect, the fruit extract includes at least two members selected from the group consisting of pomegranate extract, bilberry extract, elderberry extract, blueberry extract, Saskatoon berry extract, and the like, including combinations thereof. In yet another aspect, the fruit extract includes pomegranate extract, bilberry extract, and elderberry extract. In a further aspect, the fruit extract includes pomegranate extract, bilberry extract, elderberry extract, and blueberry extract. In yet a further aspect, the fruit extract includes pomegranate extract, bilberry extract, elderberry extract, and Saskatoon berry extract. It should be understood that the term of fruit extract can include an extract of varying amounts of solid and/or juice material. For example, in one aspect a fruit extract can include substantially liquid juice. In another aspect, a fruit extract can include both liquid juice and solid fruit matter or material. In yet another aspect, a fruit extract can include substantially solid fruit material. In some aspects the substantially solid fruit material can be a dried fruit extract. In addition to those described above, various other fruit extracts can be include in the present compositions to enhance the digestive and/or nutritional qualities of the composition. It should be noted that the term “solid” as used in the present specification refers to the non-liquid portions of a fruit or a fruit extract.

In addition to those described above, additional extracts are contemplated. Such extracts can include, without limitations, acai berry, peppermint, ginger, licorice, raspberries, black berries, cherries, aronia berries, boysenberries, loganberries, grape extracts, extracts including resveratrol, papaya, goji, cranberry, lingonberries, currents, stevia, and the like.

Various amounts of fruit extract in the composition are contemplated and can vary depending on the compositional design of the product and the intended results. Any amount of fruit extract or fruit extract combination that achieves a desired result should be considered to be within the present scope. In one aspect, for example, an effective amount of the fruit extract is included in the composition. In some aspects, the amount of fruit extract can be described for a solid dosage form, such as, for example, a tablet. In one aspect, an amount of fruit extract in a solid dosage form is from about 0.25% w/w to about 75% w/w. In another aspect, an amount of fruit extract in the composition is from about 0.5% w/w to about 50.0% w/w. In a further aspect, an amount of fruit extract in the composition is from about 0.5% w/w to about 15.0% w/w. In yet a further aspect, an amount of fruit extract in the composition is from about 0.5% w/w to about 10.0% w/w. In another aspect, an amount of fruit extract in the composition is from about 0.5% w/w to about 5.0% w/w. In yet another aspect, an amount of fruit extract in the composition is about 1.0% w/w. In a further aspect, an amount of fruit extract in the composition is about 3.0% w/w. It should be noted that an effective amount of fruit extract can include reference to a single fruit extract in the composition, or it can include reference to a combined sum of all fruit extracts the composition.

In other aspects, the amount of fruit extract can be described for a liquid dosage form, such as, for example, a drinkable liquid. In such cases, the amount of fruit extract in the composition for a liquid formulation can be from about 0.02% w/v to about 30% w/v. In another aspect, an amount of fruit extract in the composition is from about 0.04% w/v to about 10.0% w/v. In a further aspect, an amount of fruit extract in the composition is from about 0.04% w/v to about 5.0% w/v. In yet a further aspect, an amount of fruit extract in the composition is from about 0.04% w/v to about 1.0% w/v. In yet another aspect, an amount of fruit extract in the composition is about 0.1% w/v. In a further aspect, an amount of fruit extract in the composition is about 0.3% w/v. It should be noted that an effective amount of fruit extract can include reference to a single fruit extract in the composition, or it can include reference to a combined sum of all fruit extracts the composition.

Regarding the amounts of specific single fruit extracts the composition, any amount of each fruit extract that is an effective amount and thus achieves a desired result is considered to be within the present scope. In one aspect, for example, an amount of pomegranate extract in the composition for a solid dosage form is from about 0.5% w/w to about 15.0% w/w. In another aspect, an amount of pomegranate extract in the composition for a solid dosage form is from about 1.0% w/w to about 5.0% w/w. One non-limiting example of a pomegranate extract can include P-135 Pomegranate Extract (5%) available from by Naturex Inc., South Hackensack N.J. With respect to bilberry extract, in one aspect an amount in the composition for a solid dosage form is from about 0.5% w/w to about 15.0% w/w. In another aspect, an amount of bilberry extract in the composition for a solid dosage form is from about 1.0% w/w to about 5.0% w/w. One non-limiting example of a bilberry extract can include B-005 Bilberry Extract (4:1) available from BattleChem Distribution Inc., San Clemente, Calif. With respect to elderberry extract, in one aspect an amount in the composition for a solid dosage form is from about 0.5 w/w to about 15.0% w/w. In another aspect, an amount of elderberry extract in the composition for a solid dosage form is from about 1.0% w/w to about 5.0 w/w. One non-limiting example of an elderberry extract can include E-035 Elderberry Extract (5%) available from by Naturex Inc., South Hackensack N.J. With respect to blueberry extract, in one aspect an amount in the composition for a solid dosage form is from about 0.5% w/w to about 15.0% w/w. In another aspect, an amount of blueberry extract in the composition for a solid dosage form is from about 1.0% w/w to about 5.0% w/w. One specific example of a blueberry extract can be Blueberry Antioxidant Support, available from HerbaSway®, Wallingford, Conn. With respect to Saskatoon berry extract, in one aspect an amount in the composition for a solid dosage form is from about 0.5% w/w to about 15.0% w/w. In another aspect, an amount of Saskatoon berry extract in the composition for a solid dosage form is from about 1.0% w/w to about 5.0% w/w. One non-limiting example of a Saskatoon berry extract can be obtained from Prairie Berries, Keeler, SK, Canada.

For liquid formulations, individual fruit extracts can be added in various amounts. For example, in one aspect any individual fruit extract can be included in the liquid formulation in an amount of from about 0.04% w/v to about 10% w/v. In another aspect, any individual fruit extract can be included in the liquid formulation in an amount of from about 0.06% w/v to about 5% w/v.

In one specific aspect, an antacid composition can be formulated as a chewable tablet, and can include a therapeutically effective amount of magnesium citrate, a therapeutically effective amount of calcium citrate, an effective amount of blueberry fiber, an effective amount of pomegranate extract, an effective amount of bilberry extract, an effective amount of elderberry extract. In some aspects, an effective amount of blueberry extract can also be included in the formulation. One exemplary formulation can include 15.0% w/w of calcium citrate, 5.0% w/w of magnesium citrate, 20% w/w of blueberry fiber, 1.0% w/w of elderberry extract (5%), 1.0% w/w of bilberry extract (4:1), and 1.0% w/w of pomegranate extract (5%). Another exemplary formulation can include 3.0% w/w of calcium citrate, 1.0% w/w of magnesium citrate, 20% w/w of blueberry fiber, 1.0% w/w of elderberry extract (5%), 1.0% w/w of bilberry extract (4:1), and 1.0% w/w of pomegranate extract (5%). In some cases, 1.0% w/w of blueberry extract and/or Saskatoon berry extract can also be included.

In addition to the ingredients described above, other herbal and botanical extracts may be incorporated into the composition, which impart positive health benefits. In one aspect, herbal and botanical extracts for inclusion in the present formulation can be selected from, but not limited to, Ginseng, Ginko Biloba, Hawthorn berry, Saw Palmetto, Kava Kava, Echinacea, Licorice Root, Grape seed, Chammomile, Hempseed, Aloe Vera, Cinnamon Bark, Dong Quai, Gum Acacia, Cordyceps, Ho Shou Wu, Dandelion, Gynostemma, St. John's Wort, mushroom, Notginseng, Dan Shen, Noni, Rose Hips, Stevia, Garlic, Nopal, Valerian, Milk Thistle, Fenugreek, and mixtures thereof. In one specific aspect, Fenugreek can be included in the composition.

Other ingredients can include water soluble vitamins such as vitamin B1, B2, B3, B5, B6, B12, biotin, choline, folic acid, inositol, para-aminobenzoic acid (PABA), and vitamin C, oil soluble vitamins including vitamin A, vitamin D, vitamin E, and vitamin K, as well as amino acids, ionic minerals, and naturally occurring anti-oxidants. Examples of amino acids include alanine, arginine, carnitine, gamma-aminobutyric acid (GABA), glutamine, glycine, histidine, lysine, methionine, N-acetyl cysteine, ornithine, phenylalanine, taurine, tyrosine, valine, and combinations thereof. Additionally, in one aspect a probiotic can be included in the composition. In another aspect, a calcium supplement can be included in the composition. One non-limiting example of a calcium supplement is coral calcium. In one aspect, vitamin D3 can be included in the composition.

The compositions of the present invention can be formulated into a variety of dosage forms. Non-limiting examples of such dosage forms can include a tablet, a chewable tablet, a capsule, a gel capsule, a powder, a liquid, a syrup, an elixir, a suspension, a chewable gum, an effervescent beverage, a soft chew, confections, candies, bars, lozenges, and combinations thereof. The manufacture of such dosage forms is well known, and one of ordinary skill in the art, once in possession of the present disclosure, could readily formulate the present compositions and to one of these dosage forms. In one specific aspect, the composition is formulated as a chewable tablet. In another specific aspect, the composition is formulated as a drinkable liquid.

Numerous excipients are also contemplated, and various excipients can be included in the compositions of the present invention depending on the particular dosage form. Exemplary excipients can include, for example, stabilizers, preservatives, flavoring agents, thickeners, etc. Non-limiting examples of specific excipients include xanthan gum, sodium benzoate, natural and artificial flavorings, pectin, and the like. The excipients can be present individually or in combinations. Other ingredients can be included, such as sweeteners, colorants, and effervescent causing ingredients. Non-limiting examples of other ingredients can include malic acid, MCC Nutrisolve, fructose, stearic acid, magnesium stearate, silicon dioxide, and the like.

Additional ingredients are also contemplated that can be added to the formulations according to aspects of the present disclosure, and the addition of any such ingredient for any purpose is deemed to be within the present scope. Various ingredients can be utilized in any formulation type for which they provide a benefit. Non-limiting examples can include ingredients such as calcium gluconate, calcium lactate, calcium carbonate, and the like.

While such ingredients can be utilized in any formulation type (e.g. chewable, effervescent, liquid, etc.), these can be particularly useful in liquid formulations.

In another aspect of the present disclosure, a composition is contemplated whereby the blueberry ingredients are replaced with Saskatoon berry ingredients. For example, any blueberry fruit, fiber, or extract in a liquid or solid composition is replaced with an equivalent amount of Saskatoon berry fruit, fiber, or extract. Additionally, in some aspects additional blueberry ingredients can be added to such a composition.

It is to be understood that the above-described compositions and methods are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in materials, temperature, function, order, and manner of operation, assembly and use may be made without departing from the principles and concepts set forth herein.

1. An antacid composition suitable for delivery to a subject for reducing a digestive condition, comprising:
an effective amount of at least one citrate compound;
an effective amount of a blueberry product selected from the group consisting of blueberry fiber, blueberry fruit, and combinations thereof; and
an effective amount of fruit extract including a member selected from the group consisting of pomegranate extract, bilberry extract, elderberry extract, blueberry extract, and combinations thereof.

2. The composition of claim 1, wherein the citrate compound is selected from the group consisting of magnesium citrate, calcium citrate, sodium citrate, potassium citrate, and combinations thereof.

3. The composition of claim 1, wherein the citrate compound includes magnesium citrate and calcium citrate.

4. The composition of claim 1, wherein the fruit extract includes at least two members selected from the group consisting of pomegranate extract, bilberry extract, elderberry extract, blueberry extract, and combinations thereof.

5. The composition of claim 1, wherein the fruit extract includes pomegranate extract, bilberry extract, and elderberry extract.

6. The composition of claim 5, wherein the fruit extract includes blueberry extract.

7. The composition of claim 1, wherein the blueberry product is blueberry fiber and the composition is formulated as a member selected from the group consisting of a tablet, a capsule, a powder, a gum, an effervescent tablet, a soft chew, and combinations thereof

8. The composition of claim 7, wherein the composition is formulated as a tablet.

9. The composition of claim 8, wherein the effective amount of citrate compound is from about 1.0% w/w to about 75.0% w/w.

10. The composition of claim 8, wherein the citrate compound includes magnesium citrate and calcium citrate, the effective amount of magnesium citrate is from about 0.5% w/w to about 15.0% w/w, and the effective amount of calcium citrate is from about 0.5% w/w to about 40.0% w/w.

11. The composition of claim 8, wherein the effective amount of blueberry fiber is from about 1.0% w/w to about 75.0% w/w.

12. The composition of claim 8, wherein the effective amount of fruit extract is from about 0.5% w/w to about 15.0% w/w.

13. The composition of claim 1, wherein the blueberry product is blueberry fruit and the composition is formulated as a member selected from the group consisting of a liquid, a syrup, an elixir, a suspension, an effervescent beverage, and combinations thereof.

14. The composition of claim 13, wherein the composition is formulated as a liquid.

15. The composition of claim 14, wherein the effective amount of citrate compound is from about 0.1% w/v to about 20% w/v.

16. The composition of claim 14, wherein the citrate compound includes magnesium citrate and calcium citrate, the effective amount of magnesium citrate is from about 0.05% w/v to about 10% w/v, and the effective amount of calcium citrate is from about 0.1% w/v to about 20% w/v.

17. The composition of claim 14, wherein the effective amount of blueberry fruit is from about 0.1% w/v to about 10% w/v.

18. The composition of claim 14, wherein the effective amount of fruit extract is from about 0.02% w/v to about 30% w/v.

19. The composition of claim 1, further comprising vitamin D.

20. An antacid composition suitable for delivery to a subject and formulated as a chewable tablet, comprising:
a therapeutically effective amount of magnesium citrate;
a therapeutically effective amount of calcium citrate;
an effective amount of blueberry fiber;
an effective amount of pomegranate extract;
an effective amount of bilberry extract;
an effective amount of elderberry extract; and
an effective amount of blueberry extract.

21. A method of treating a digestive condition, comprising orally ingesting the composition of claim 20.

22. An antacid composition suitable for delivery to a subject for reducing a digestive condition, comprising an effective amount of at least two citrate compounds.

23. An antacid composition suitable for delivery to a subject for reducing a digestive condition, comprising:
an effective amount of at least one citrate compound;
an effective amount of a Saskatoon berry product selected from the group consisting of Saskatoon berry fiber, Saskatoon berry fruit, and combinations thereof; and
an effective amount of fruit extract including a member selected from the group consisting of pomegranate extract, bilberry extract, elderberry extract, Saskatoon berry extract, and combinations thereof.
